(19) United States
(12) Patent Application Publication
(10) Pub. No.: US 2024/0257413 A1
(43) Pub. Date: Aug. 1, 2024

(54) IMAGE PROCESSING APPARATUS, IMAGE PROCESSING METHOD, AND NON-TRANSITORY COMPUTER-READABLE MEDIUM STORING PROGRAM

(71) Applicant: TOKYO INSTITUTE OF TECHNOLOGY, Meguro-ku, Tokyo (JP)

(72) Inventors: Kenji SUZUKI, Meguro-ku, Tokyo (JP); Ze JIN, Meguro-ku, Tokyo (JP)

(21) Appl. No.: 18/546,940

(22) PCT Filed: Mar. 2, 2022
(86) PCT No.: PCT/JP2022/008813

§ 371 Date: Apr. 16, 2024

(30) Foreign Application Priority Data
Mar. 2, 2021 (JP) 2021-032652

(51) Int. Cl. G06T 11/00

(52) U.S. Cl. CPC G06T 11/006; G06T 2210/41; G06T 2211/441

(57) ABSTRACT

The present disclosure makes it possible to acquire an image of high quality while shorten an imaging time by an MRI device. A data acquisition unit (11) acquires low-density sampling k-space raw data obtained by imaging a subject while reducing the number of measurement points by the MRI device, and outputs input data (IN) based on the low-density sampling k-space raw data. An inference processing unit (12) performs inference processing by inputting the input data (IN) to a trained model, and outputs restored image data (IMG) in which the image quality reduced by reducing the number of measurement points has been restored. An image display unit (13) displays the restored image data (IMG).

### TECHNICAL FIELD

The present disclosure relates to an image processing apparatus, and an image processing method, and a non-transitory computer-readable medium storing a program.

### BACKGROUND ART

A magnetic resonance imaging (MRI) apparatus is an apparatus that acquires two-dimensional or three-dimensional image information by applying a magnetic field to a living body serving as a subject, such as a tissue of a human body, and using a resulting nuclear magnetic resonance (NMR) phenomenon. The MRI apparatus has excellent features, such as the capability of imaging tissues that cannot be imaged by a computed tomography (CT) apparatus and the absence of radiation exposure.

When reading the NMR signal during imaging in the MRI, a gradient magnetic field is applied to the subject to encode a position, and imaging data is acquired as a set of raw data in the k-space, which is a measurement space. Since the k-space and the real space are in a Fourier transform relationship with each other, an image in the real space is reconstructed by performing the Fourier transform on the imaging data in the k-space (Patent literatures 1 to 3).

The imaging of the subject in the MRI apparatus takes a relatively long time and generally requires several tens of minutes of imaging time. Therefore, to reduce a burden on the subject due to the long time required for imaging and to improve the imaging throughput in the MRI apparatus, a high-speed imaging technique is used in which measurement points in the k-space are thinned out and sampled, and unmeasured data between the measurement points is interpolated to compensate for a resolution of the image. Compressed sensing is known as an example of the high-speed imaging technique. For example, fast spin echo (FSE), echo planner imaging (EPI), and gradient echo (GE) are known as other high-speed imaging techniques.

### CITATION LIST

### Patent Literature

Patent Literature 1: Japanese Unexamined Paten Application Publication No. 2017-000573
Patent Literature 2: Japanese Unexamined Paten Application Publication No. 10-277000
Patent Literature 3: Japanese Unexamined Paten Application Publication No. 2001-218752
Patent Literature 4: United States Patent No. 7545965
Patent Literature 5: United States Patent No. 6819790

### SUMMARY OF INVENTION

### Technical Problem

However, since a ratio of the measurement points that can be thinned out are limited with the general high-speed imaging method described above, the imaging time that can be shortened is only about 50% compared to that of the normal imaging. Therefore, it is difficult to significantly improve the throughput in principle.

Further, there are problems in which the thinning out of measurement points causes a decrease in the resolution of the reconstructed image, an increase in artifacts, and an increase in noise. Accordingly, the quality of the image deteriorates.

FIG. 28 shows examples of k-space raw data imaged by normal high-definition imaging and high-speed imaging by an MRI apparatus, and a reconstructed image. In displaying the k-space raw data, the vertical axis denotes phase encoding and the horizontal axis denotes frequency encoding.

In this example, since measurement of an entire range of phase encoding to be measured is performed in the high-definition imaging on the upper side, the image reconstructed from the high-definition imaging is a high-definition image. However, as described above, the imaging takes a relatively long time due to a large number of measurement points.

On the other hand, in the high-speed imaging on the lower side, imaging at a position indicated by s is skipped, and a blacked-out part in the k-space raw data corresponds to the thinned-out measurement points. In this example, the measurement points are reduced to approximately ½ compared to the high -definition imaging. In the image reconstructed from the k-space raw data, the image quality is degraded and artifacts occur compared to the example of the high-definition imaging due to missing information caused by imaging with thinning-out sampling, and, especially, blurred tissue boundaries can be seen. Therefore, if the image quality does not meet a required level, measures such as increasing measurement points are required, and the reduction of imaging time by high-speed imaging is more limited.

### Solution to Problem

An aspect of the present disclosure is an image processing apparatus including: a data acquisition unit configured to acquire lower-density sampling k-space raw data obtained by imaging a subject by thinning-out measurement points by an MRI apparatus and output input data based on the lower-density sampling k-space raw data; an estimation processing unit configured to perform estimation processing by inputting the input data into a learned model and output recovered image data whose image quality reduced by the thinned-out measurement points has been recovered; and an image display unit configured to display the recovered image data. Thus, the image quality of the input data that is reduced by the thinned-out measurement points can be recovered, and a high-quality image based on the recovered image data whose image quality has been recovered can be displayed.

An aspect of the present disclosure is the image processing apparatus described above, it is desirable in the image processing apparatus that the data acquisition unit converts the lower-density sampling k-space raw data into interpolated k-space raw data by interpolating the thinned-out measurement points and outputs the interpolated k-space raw data to the estimation processing unit as the input data, and the estimation processing unit is configured as a k-space estimation processing unit configured to perform estimation processing by inputting the interpolated k-space raw data into the learned model, output estimated k-space raw data whose image quality reduced by the thinned-out measurement points has been recovered, and output the recovered image data reconstructed by performing Fourier transform on the estimated k-space raw data. Thus, the input data that is k-space raw data can be appropriately input to the estimation processing unit.

An aspect of the present disclosure is the image processing apparatus described above, it is desirable in the image processing apparatus that the data acquisition unit acquires the interpolated k-space raw data inversely reconstructed by performing Fourier transform on image data that has been reconstructed by performing Fourier transform on the lower-density sampling k-space raw data. Thus, the lower-density sampling k-space raw data can be appropriately interpolated.

An aspect of the present disclosure is the image processing apparatus described above, it is desirable in the image processing apparatus that the data acquisition unit outputs image data obtained by performing Fourier transform on the lower-density sampling k-space raw data to the estimation processing unit as the input data, and the estimation processing unit is configured as an image-space estimation unit configured to perform estimation processing by inputting the transformed image data into the learned model and to output the recovered image data whose image quality reduced by the thinned-out measurement points has been recovered. Thus, the input data that is image data can be appropriately input to the estimation processing unit.

An aspect of the present disclosure is the image processing apparatus described above, it is desirable in the image processing apparatus that, in the estimation processing unit, one or more k-space estimation processing units and one or more image-space estimation processing units are arranged in series, the k-space estimation processing unit performs estimation processing by inputting k-space raw data, which is input data, into the learned model, outputs the k-space raw data whose image quality has been recovered, and outputs second image data reconstructed by performing Fourier transform on the k-space raw data, the image-space estimation processing unit performs estimation processing by inputting the image data, which is input data, into the learned model, and outputs image data whose image quality has been recovered, when the k-space estimation processing unit is arranged in the latter stage of the image-space estimation processing unit, an inverse image reconstruction unit is provided between the image-space estimation processing unit and the k-space estimation processing unit to output k-space raw data, which is inversely reconstructed by performing Fourier transform on image data output from the image-space estimation processing unit in the former stage, to the k-space estimation processing unit in the latter stage, and image data output from the end of serial array of the k-space estimation processing unit and the image-space estimation processing unit is output as the recovered image data. This makes it possible to perform multi -stage estimation processing and further improve the image quality of the recovered image data.

An aspect of the present disclosure is the image processing apparatus described above, it is desirable in the image processing apparatus that, when a head of the serial array of the k-space estimation processing unit and the image-space estimation processing unit is the k-space estimation processing unit, the data acquisition unit converts the lower-density sampling k-space raw data into interpolated k-space raw data by interpolating the thinned-out measurement points and outputs the interpolated k-space raw data to the head k-space estimation processing unit as the input data, and when the head of the serial array of the k -space estimation processing unit and the image-space estimation processing unit is the image-space estimation processing unit, the data acquisition unit outputs image data obtained by performing Fourier transform on the lower-density sampling k-space raw data to the head image-space estimation processing unit as the input data. Thus, when multi-stage estimation processing is performed, input data in an appropriate format can be input to the head estimation processing unit.

An aspect of the present disclosure is the image processing apparatus described above, it is desirable in the image processing apparatus that the data acquisition unit reads learning data that is lower-density sampling k-space raw data obtained in advance by imaging the subject with thinning-out the measurement points by the MRI apparatus and teacher data that is high-density sampling k-space raw data obtained in advance by imaging the subject without thinning-out the measurement points by the MRI apparatus, and outputs learning input data based on the learning data and the teacher data that have been read to the estimation processing unit, and the estimation processing unit constructs the learned model by performing supervised learning according to the learning input data. Thus, a learned model used for estimation processing can be appropriately constructed.

An aspect of the present disclosure is the image processing apparatus described above, it is desirable in the image processing apparatus that the data acquisition unit reads learning data obtained by reconstructing lower-density sampling k-space raw data obtained in advance by imaging the subject with thinning-out the measurement points by the MRI apparatus and teacher data obtained by reconstructing high-density sampling k-space raw data obtained in advance by imaging the subject without thinning-out the measurement points by the MRI apparatus, and outputs learning input data based on the learning data and the teacher data that has been read to the estimation processing unit, and the estimation processing unit constructs the learned model by performing supervised learning based on the learning input data. Thus, a learned model used for estimation processing can be appropriately constructed.

An aspect of the present disclosure is the image processing apparatus described above, it is desirable in the image processing apparatus that the learned model is a network constructed by inputting learning data and teacher data into a network configured as an MTANN (Massive-Training Artificial Neural Network) to perform learning. Thus, a learned model used for estimation processing can be appropriately constructed.

An aspect of the present disclosure is an image processing method including: acquiring lower-density sampling k-space raw data obtained by imaging a subject with thinning-out measurement points by an MRI apparatus, and outputting input data based on the lower-density sampling k-space raw data; performing estimation processing by inputting the input data into a learned model, and outputting recovered image data whose image quality reduced by the thinned-out measurement points has been recovered; and displaying the recovered image data. Thus, the image quality of the input data that is reduced by the thinned-out measurement points can be recovered, and a high-quality image based on the recovered image data whose image quality has been recovered can be displayed.

An aspect of the present disclosure is a program to cause a computer to execute: acquiring lower-density sampling k-space raw data obtained by imaging a subject with thinning-out measurement points by an MRI apparatus, and outputting input data based on the lower-density sampling k-space raw data; performing estimation processing by inputting the input data into a learned model, and outputting recovered image data whose image quality reduced by the thinned-out measurement points has been recovered; and displaying the recovered image data. Thus, the image quality of the input data that is reduced by the thinned-out measurement points can be recovered, and a high-quality image based on the recovered image data whose image quality has been recovered can be displayed.

### Advantageous Effects of Invention

According to the present disclosure, it is possible to provide an information processing apparatus, an information processing method, and a program that can acquire a high-quality image while shortening imaging time of an MRI apparatus.

### BRIEF DESCRIPTION OF DRAWINGS

FIG. 1 shows an example of a hardware configuration for realizing an image processing apparatus according to a first exemplary embodiment;

FIG. 2 shows a schematic configuration of the image processing apparatus according to the first exemplary embodiment;

FIG. 3 shows a more detailed configuration of the image processing apparatus according to the first exemplary embodiment;

FIG. 4 is a flowchart of processing of the image processing apparatus according to the first exemplary embodiment;

FIG. 5 is a diagram showing an overview of reading of k-space raw data in a data acquisition unit;

FIG. 6 is a diagram schematically showing a configuration of the image processing apparatus when the data acquisition unit has an interpolation function for lower-density k-space raw data;

FIG. 7 is a flowchart of processing procedure of a step SE11;

FIG. 8 is a diagram showing a flow of data in the image processing apparatus according to the first exemplary embodiment;

FIG. 9 is a diagram schematically showing a configuration when the image processing apparatus according to the first exemplary embodiment has a function for constructing a learned model;

FIG. 10 is a flowchart of a learning process of the image processing apparatus according to the first exemplary embodiment;

FIG. 11 is a diagram showing a structure of MTANN and an outline of inputting data to the MTANN;

FIG. 12 is a diagram schematically showing a configuration of the image processing apparatus when an estimation unit and a machine learning unit are integrated;

FIG. 13 is a diagram showing examples of images of a subject when imaged by different imaging methods;

FIG. 14 is a diagram schematically showing a configuration of an image processing apparatus according to a second exemplary embodiment ;

FIG. 15 is a flowchart of processing in an estimation phase of the image processing apparatus according to the second exemplary embodiment;

FIG. 16 is a diagram showing a flow of data in the image processing apparatus according to the second exemplary embodiment;

FIG. 17 is a diagram schematically showing a configuration when the image processing apparatus according to the second exemplary embodiment has a learned model construction function;

FIG. 18 is a flowchart of a learning process of the image processing apparatus according to second the exemplary embodiment;

FIG. 19 is a diagram schematically showing a configuration of an image processing apparatus that is a first configuration example according to a third exemplary embodiment;

FIG. 20 is a flowchart of processing in an estimation phase of the image processing apparatus that is the first configuration example according to the third exemplary embodiment;

FIG. 21 is a diagram schematically showing a configuration of an image processing apparatus that is a second configuration example according to the third exemplary embodiment;

FIG. 22 is a flowchart of processing in an estimation phase of the image processing apparatus that is the second configuration example according to the third exemplary embodiment;

FIG. 23 is a diagram schematically showing a configuration of an image processing apparatus that is a first configuration example according to a fourth exemplary embodiment;

FIG. 24 is a flowchart of processing in an estimation phase of the image processing apparatus that is the first configuration example according to the fourth exemplary embodiment;

FIG. 25 is a diagram schematically showing a configuration of an image processing apparatus that is a second configuration example according to the fourth exemplary embodiment;

FIG. 26 is a flowchart of processing in an estimation phase of the image processing apparatus that is the second configuration example according to the fourth exemplary embodiment;

FIG. 27 is a diagram schematically showing a configuration of an estimation processing unit of a filter bank image output type; and

FIG. 28 is a diagram showing examples of k-space raw data imaged by normal high-definition imaging and high-speed imaging by an MRI apparatus, and a reconstructed image.

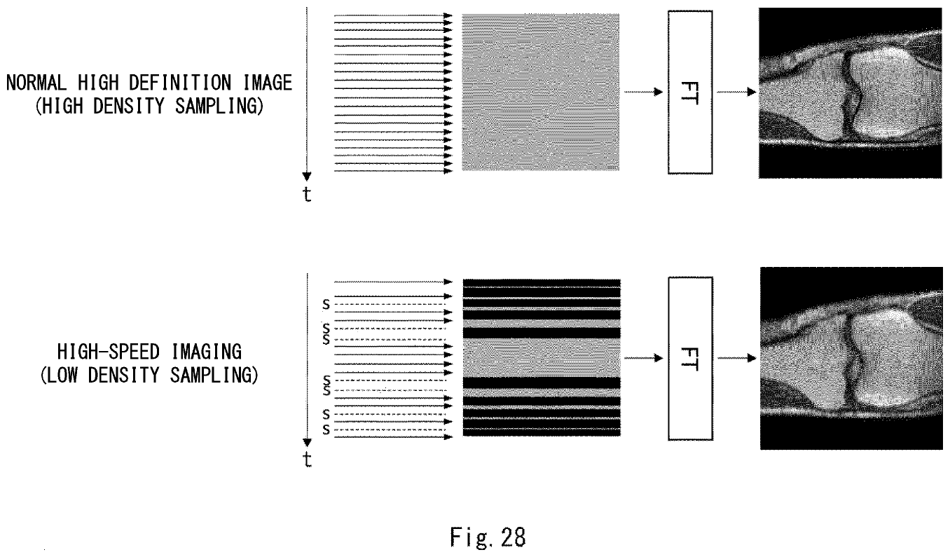

### DESCRIPTION OF EMBODIMENTS

The specific exemplary embodiments will be described with reference to the drawings. However, the present disclosure is not limited to the following exemplary embodiments. In order to clarify the description, the following descriptions and drawings will be simplified as appropriate. The same elements are denoted by the same symbols, and overlapping descriptions will be omitted.

### First Exemplary Embodiment

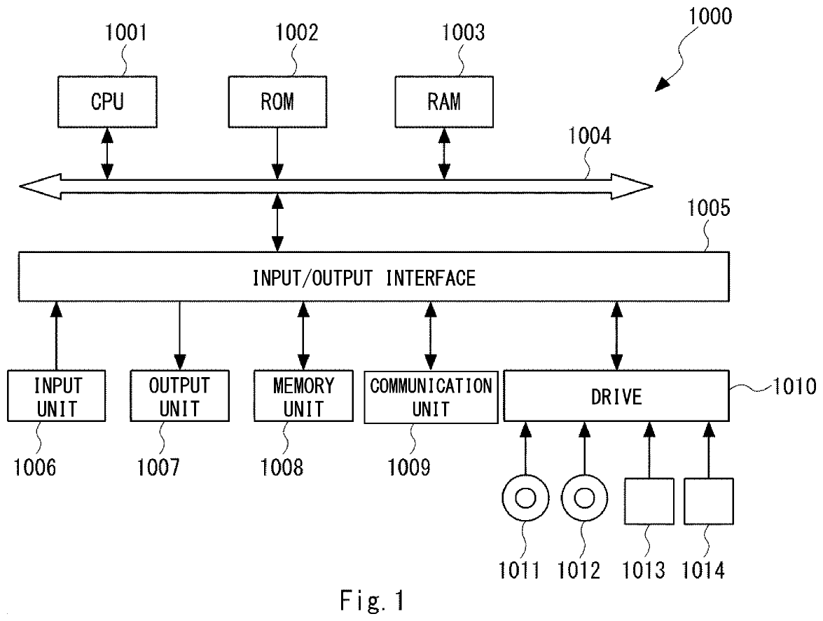

First, as a premise for understanding an image processing apparatus according to a first exemplary embodiment, an example of a hardware configuration for realizing an image processing apparatus will described. FIG. 1 shows an example of a hardware configuration for realizing the image processing apparatus according to the first exemplary embodiment. An image processing apparatus 100 can be realized by a computer 1000 such as a dedicated computer or a personal computer (PC). However, the computer need not be physically single, and may be multiple when performing distributed processing. As shown in FIG. 1, the computer 1000 includes a central processing unit (CPU) 1001, a read only memory (ROM) 1002 and a random-access memory (RAM) 1003, which are connected to each other via a bus 1004. Although an explanation of an OS software for operating the computer will be omitted, it is assumed that the computer for constructing the image processing apparatus also naturally has one.

An input/output interface 1005 is also connected to the bus 1004. For example, an input unit 1006 configured of a keyboard, mouse, sensor, or the like, a display configured of a CRT, LCD, or the like, an output unit 1007 configured of a headphone, speaker, or the like, a memory unit 1008 configured of a hard disk, or the like, and a communication unit 1009 configured of a modem, terminal adapter, or the like, are connected to the input/output interface 1005.

The CPU 1001 performs various types of processing according to various programs stored in the ROM 1002 or loaded into the RAM 1003 from the memory unit 1008, and in the present exemplary embodiment, for example, processing of various parts of the image processing apparatus 100 described later. A GPU (Graphics Processing Unit) may be provided to perform various types of processing according to various programs stored in the ROM 1002 or loaded into the RAM 1003 from the memory unit 1008, as in the CPU 1001, and in the present exemplary embodiment, for example, processing of various parts of the image processing apparatus 100 described later. The GPU is suitable for applications in which routine processing is performed in parallel, and by applying it to processing in neural networks, which will be described later, it is possible to improve the processing speed compared to the CPU 1001. Data necessary for the CPU 1001 and the GPU to perform various types of processing are also stored in the RAM 1003 as appropriate.

For example, the communication unit 1009 performs communication processing via the Internet (not shown), transmits data provided by the CPU 1001, and outputs data received from the communication partner to the CPU 1001, the RAM 1003, and the memory unit 1008. The storage unit 1008 communicates with the CPU 1001 to store and erase information. The communication unit 1009 also performs processing for communicating analog or digital signals with other devices.

Further, the input/output interface 1005 is appropriately connected to a drive 1010, for example, a magnetic disk 1011, an optical disk 1012, a flexible disk 1013, or a semiconductor memory 1014 is appropriately provided to the input/output interface 1005, and a computer program read from them is appropriately installed in the memory unit 1008.

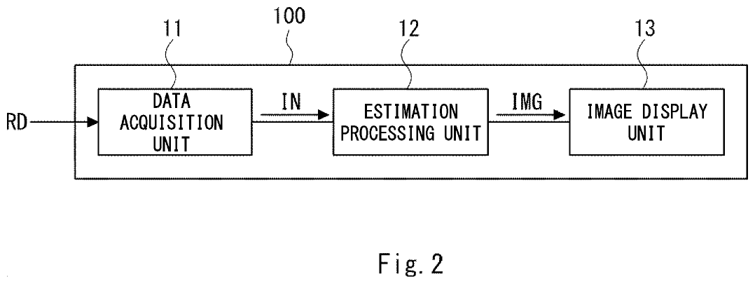

A configuration and operation of the image processing apparatus 100 according to the first exemplary embodiment will be described below. FIG. 2 schematically shows the configuration of the image processing apparatus 100 according to the first exemplary embodiment. The image processing apparatus includes a data acquisition unit 11, an estimation processing unit 12 and an image display unit 13.

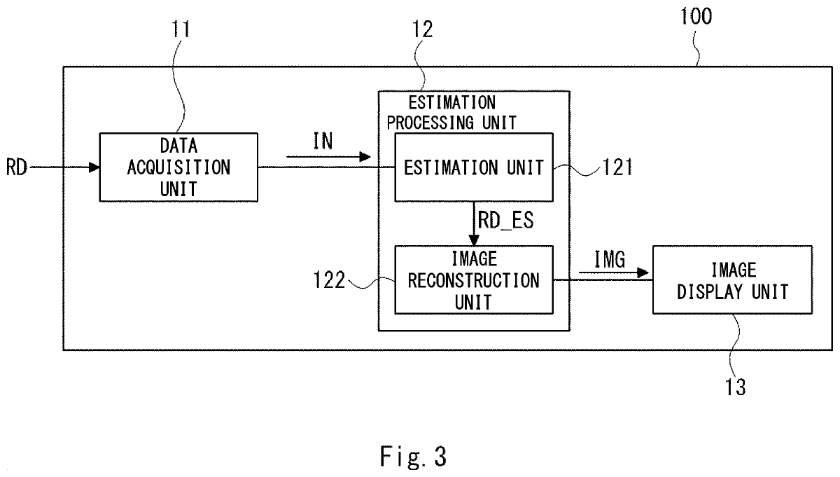

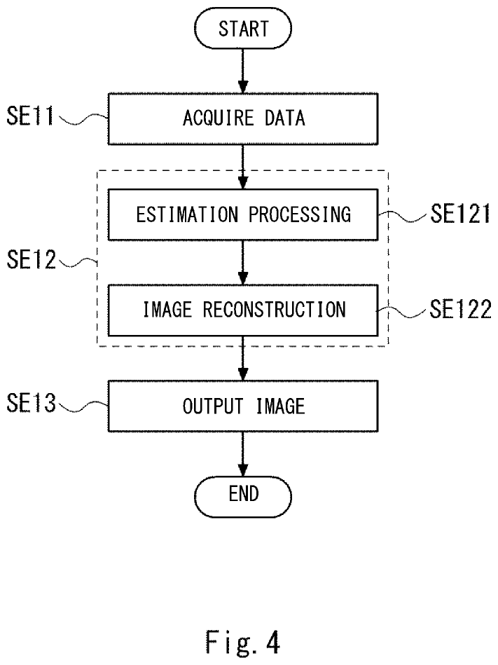

FIG. 3 shows the configuration of the image processing apparatus 100 according to the first exemplary embodiment in more detail. FIG. 4 shows a flow chart of processing of the image processing apparatus 100 according to the first exemplary embodiment. The data acquisition unit 11 reads k-space raw data RD that is k-space raw data obtained by performing high-speed imaging with thinning-out of measurement points by an MRI apparatus. In the following, the k-space raw data obtained by performing the high-speed imaging with thinning-out of measurement points by the MRI apparatus, that is, obtained by lower-density sampling, is also referred to as lower-density sampling k-space raw data. The data acquisition unit 11 outputs the read lower-density sampling k-space raw data RD to the estimation processing unit 12. The data acquisition unit 11 can read the lower-density sampling k-space raw data RD from a memory device or the MRI apparatus as appropriate (step SE11 in FIG. 4).

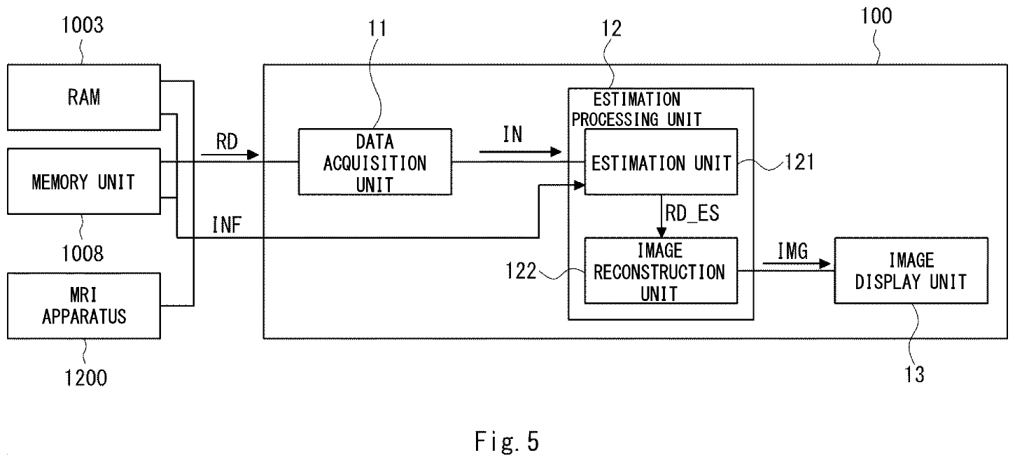

FIG. 5 shows an overview of acquisition of the k-space raw data performed by the data acquisition unit 11. For example, the memory unit 1008 and RAM 1003 described above can be used as the memory device. The lower-density sampling k-space raw data RD is stored in the memory device such as the memory unit 1008 and RAM 1003, and can be read by the data acquisition unit 11 as appropriate. The lower-density sampling k-space raw data RD may also be provided from the MRI apparatus 1200 to the data acquisition unit 11.

When the k-space raw data is provided from the data acquisition unit 11 to the estimation processing unit 12 as input data IN, to smoothly perform estimation processing performed by the estimation processing unit 12 or learning processing described later with high accuracy, it is desirable to provide the k-space raw data in which the thinned-out measurement points are interpolated, instead of the lower-density sampling k-space raw data RD obtained by performing the high-speed imaging with thinning-out of measurement points. Therefore, it is desirable that the data acquisition unit 11 has a function to interpolate the thinned-out measurement points in the lower-density sampling k-space raw data RD.

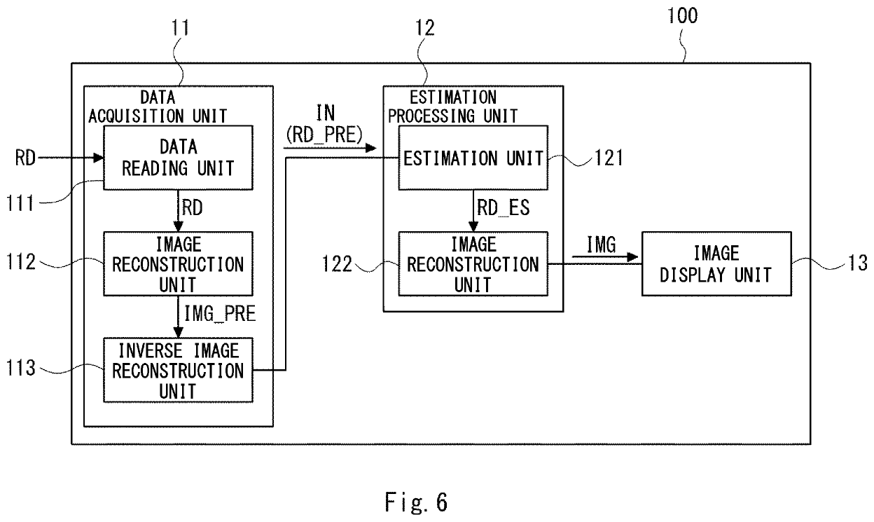

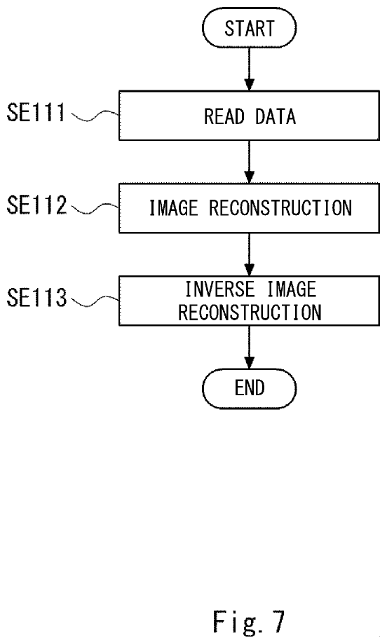

FIG. 6 schematically shows a configuration of the image processing unit 100 when the data acquisition unit has an interpolation function for the lower-density sampling k-space raw data RD. FIG. 7 shows a flowchart of a processing procedure in the step SE11. As shown in FIG. 6, the data acquisition unit 11 includes a data reading unit 111, an image reconstruction unit 112, and an inverse image reconstruction unit 113. The image reconstruction unit 112 and the inverse image reconstruction unit 113 constitute a data interpolation unit that interpolates the thinned-out measurement points in the lower-density sampling k-space raw data RD.

The data reading unit 111 is configured to read data such as the lower-density sampling k-space raw data RD (step SE111 in FIG. 7). The image reconstruction unit 112 performs Fourier-transform on the lower-density sampling k-space raw data RD to reconstruct interpolated image data IMG_PRE (step SE112 in FIG. 7). The inverse image reconstruction unit 113 performs inverse Fourier-transform on the interpolated image data IMG_PRE to obtain interpolated k-space raw data RD_PRE and outputs the interpolated k-space raw data RD_PRE to the estimation processing unit 12 as the input data IN (step SE113 in FIG. 7).

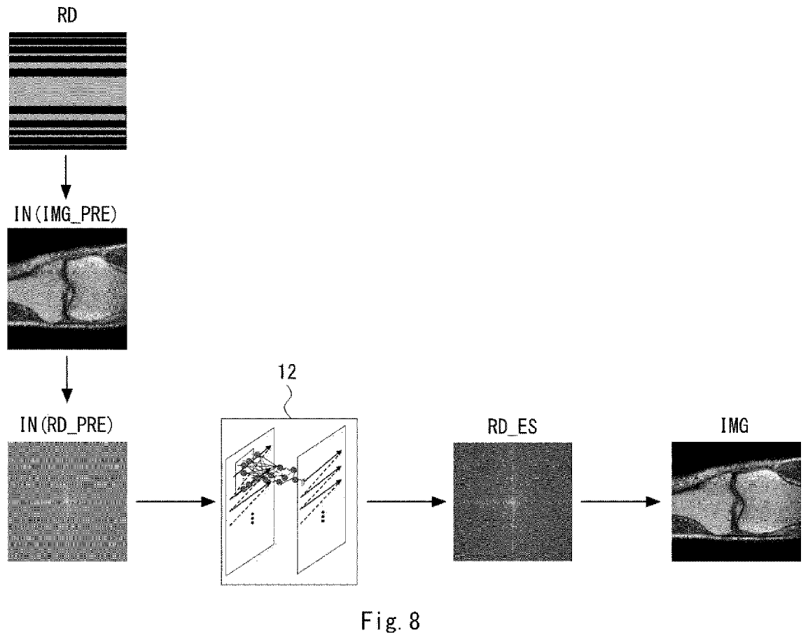

Next, interpolation of the lower-density sampling k-space raw data RD by the image reconstruction unit 112 and the inverse image reconstruction unit 113 will be described. FIG. 8 shows a flow of data in the image processing apparatus 100 according to the first exemplary embodiment. For the k-space raw data represented in FIG. 8, the vertical axis denotes phase-encoding, the horizontal axis denotes frequency-encoding. First, since the lower-density sampling k-space raw data RD is a result of measurement with thinning-out of measurement points, measurement in certain phase encoding is missing and missing parts are represented as a black-filled part.

When the lower-density sampling k-space raw data RD is reconstructed as it is into an image by the image reconstruction unit 112, the interpolated image data IMG_PRE is obtained as a low-resolution image with image quality deterioration due to the missing measurement points. Then, the inverse image reconstruction unit 113 performs the Fourier-transform on the interpolated image data IMG_PRE, the interpolated k-space raw data RD_PRE in which the phase and frequency encoding information of the interpolated image data IMG_PRE is expanded into the k-space is obtained. In this case, although the phase and frequency encoding information of the low-resolution interpolated image data IMG_PRE is reflected in the interpolated k-space raw data RD_PRE, data in a state in which no measurement points are missing is obtained. In this way, the missing measurement points can be interpolated by sequentially performing the image reconstruction processing and image inverse conversion processing on the k-space raw data in which the measurement points are thinned-out.

In the sampling method, sampling can be skipped at regular intervals. However, to perform more efficient and effective lower-density data collection, parts with higher image information and image energy are sampled at a higher density, while other parts are sampled at lower density. High-density sampling is performed in a low-frequency region, i.e., near the center of k-space coordinates, because there is more image energy and information, while lower-density sampling is performed in the high-frequency region away from the center.

It goes without saying that the interpolation of the k-space raw data in which the measurement points are thinned-out described here is merely an exemplification, and various other interpolation methods may be used.

The estimation processing unit 12 includes an estimation unit 121 and an image reconstruction unit 122, and performs processing according to the procedure of a step SE12 including steps SE121 and SE122. The estimation unit 121 holds a learned model that has been constructed in advance. The estimation unit 121 inputs the lower-density sampling k-space raw data RD or the interpolated k-space raw data RD_PRE as the input data IN to be analyzed into the held learned model, and outputs estimated k-space raw data RD_ES estimated based on the learning result (step SE121 in FIG. 4). The estimation here means that input data corresponding to the low-quality image is input into the learned model, and the data whose image quality has been recovered is output. The image reconstruction unit 122 performs Fourier-transform on the estimated k-space raw data RD_ES to reconstruct recovered image data IMG of the subject whose image quality has been improved. The reconstructed recovered image data IMG is output to the image display unit 13 (step SE122 in FIG. 4).

The estimation processing unit 12 is also referred to as a k-space estimation processing unit because the k-space raw data is input as input data to perform estimation processing.

The estimation unit 121 may receive information INF indicating the learned model from an external memory device or the like and hold the learned model based on the information INF indicating the received learned model, as shown in FIG. 5. The information INF may be provided in the form of, for example, a parameter specifying a weighting factor between neurons of a neural network constituting the learned model.

Based on the recovered image data IMG received from the estimation processing unit 12, the image display unit 13 displays the image to be used for examination on a display device such as a display (step SE13 in FIG. 4). For example, the output unit 1007 described above may be used as the image display unit 13.

While the configuration and operation (i.e., estimation phase) related to the estimation and image reconstruction using the learned model in the estimation processing unit 12 have been described above, the estimation processing unit 12 may further include a machine learning unit for constructing the learned model.

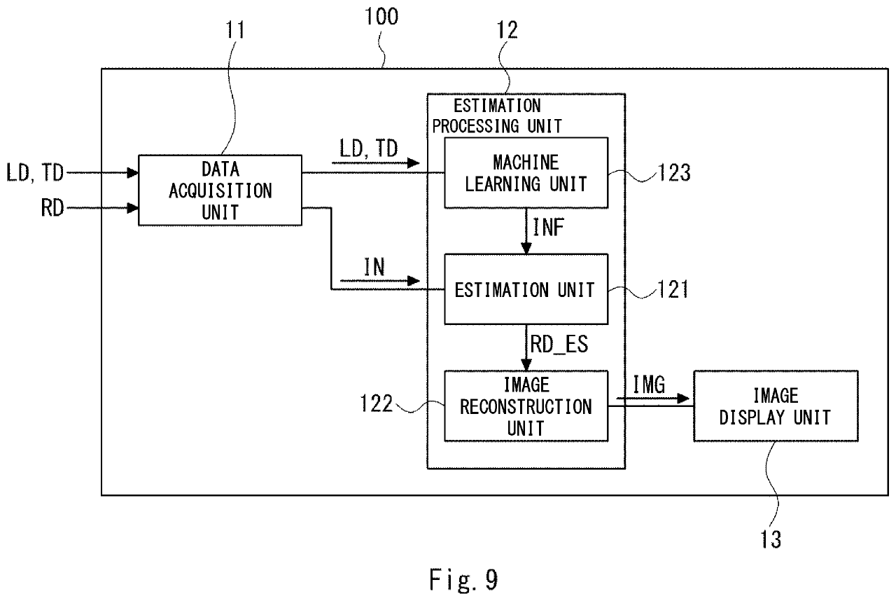

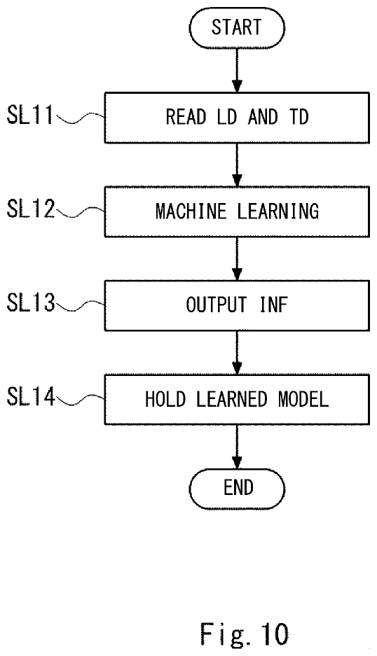

FIG. 9 schematically shows a configuration when the image processing unit 100 according to the first exemplary embodiment has a function for constructing a learned model. FIG. 10 shows a flow chart of learning process of the image processing apparatus 100 according to the first exemplary embodiment. In FIG. 9, the estimation processing unit 12 further includes a machine learning unit 123.

The machine learning unit 123 is configured to learn data read from a memory device by supervised learning in a learning phase. In the learning phase, the data acquisition unit 11 reads learning data LD and teacher data TD that are imaged by the MRI apparatus in advance and stored in a memory device such as a RAM 1003 or a memory unit 1008, and outputs the learning data LD and teacher data TD to the machine learning unit 123 (step SL11 in FIG. 10).

The machine learning unit 123 inputs the received learning data LD and the teacher data TD into a neural network to perform machine learning by deep learning (step SL12 in FIG. 10). The learning data LD is lower-density sampling k-space raw data obtained in advance by performing high-speed imaging of the subject with thinning-out of measurement points. The teacher data TD is high -density sampling k-space raw data obtained in advance by performing high-definition imaging of the subject without thinning-out of measurement points.

In the present exemplary embodiment, it is desirable to use a method for directly learning images such as image output-type deep learning as a machine learning method for raw image data. An example of image output-type deep learning is Massive-Training Artificial Neural Network (MTANN). The MTANN is a nonlinear deep learning model that can output images. In the present exemplary embodiment, the machine learning unit 123 learns a learned model (estimator) obtained by the MTANN.

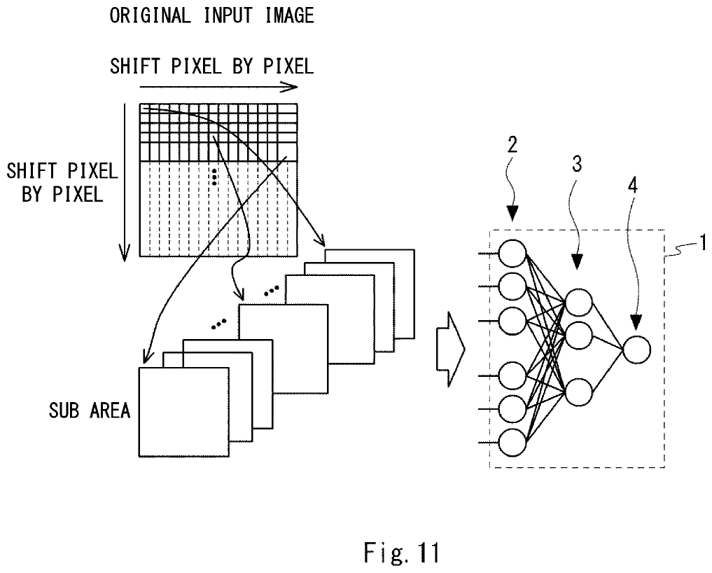

The MTANN is described in Patent Literatures 4 and 5, and will be briefly described below. The MTANN is a neural network applicable to various image processing and pattern recognition processing. FIG. 11 shows a structure of the MTANN and an outline of data input to the MTANN.

The MTANN 1 includes a multi-layer neural network (Artificial Neural Network: ANN) that can directly manipulate a level of an input pixel and a level of an output pixel. The multilayer ANN of the MTANN 1 includes of an input layer 2, a hidden layer 3, and an output layer 4. The input layer 2 and the hidden layer 3 includes a plurality of units (neurons), while the output layer 4 includes only one unit (neuron).

The input layer 2, the hidden layer 3, and the output layer 4 have a linear function, a sigmoid function, and a linear function as activation functions, respectively. Since the characteristics of the ANN in image processing are greatly improved by using the linear function as the activation function of the output layer, the linear function is applied as the activation function of the unit of the output layer instead of the sigmoid function in the MTANN.

The MTANN 1 can obtain various filtering functions such as high-pass filtering, low-pass filtering, bandpass filtering, noise reduction, edge enhancement, edge detection, interpolation, pattern matching, object enhancement, object recognition, wavelet transform, texture analysis and segmentation by Fourier-transform through learning, and can perform image processing and pattern recognition. This makes it possible for the MTANN 1 to approximate any mapping operation.

When inputting an image to the MTANN in the learning and execution phases, pixel values of the input image are normalized. For example, when a quantization level of the pixel value of the input image is 10 bits (1024 gray levels), the pixel value is normalized in such a manner it is 0 when the pixel value is 0 (lower limit value of a dynamic range) and it is 1 when the pixel value is 1023 (upper limit value of the dynamic range). This normalization is an exemplification, and other normalization methods may be used.

Input of the image to the MTANN 1 is performed by sequentially inputting subregions, which are obtained by scanning an original input image using a local window R_{S} having a predetermined size. At this time, for example, the input image is scanned by repeating process of shifting the local window R_{S} from one end of the row of pixels by one pixel, and shifting it to the next row when it reaches the other end. That is, the local window is shifted by one pixel while having overlap. Thus, after subregions are cut out from one piece of the input data and the pixel values included therein are normalized, the subregions can be input into the MTANN 1.

The pixel value f(x, y) output from the MTANN 1 is a continuous value corresponding to the pixel value in the center of the local window R_{S} , and is expressed by the following expression:

$$f ( x , y ) = NN \{ I ( x , y ) \} = NN \{ g ( x - i , y - j ) \vert i , j ∈ {R}_{S} \} \qquad [ 1 ]$$

In the expression [1], x and y are the coordinates of the image, NN{*} is an output of a deformed ANN, I(x, y) is an input vector to the deformed ANN, and g(x, y) is the normalized pixel value in the local window R_{S}.

The pixel value f(x, y) of the output image, that is, the output value of the MTANN, is output as an estimate value of a desired value according to a field of application. When a low-quality image is converted into a high-quality image as in the present exemplary embodiment, the pixel value f(x, y) is output as the estimate value of the pixel value of the high-quality image. When identifying whether an image contains a lesion or not, likelihood indicating the lesion is output as the pixel value f(x, y).

The MTANN 1 is trained by performing supervised learning using teacher data as disclosed in Patent Literatures 4 and 5. Then, by inputting an input image to be diagnosed into the trained MTANN, the above estimated pixel values are obtained for each local window R_{S} . Then, a desired output image can be obtained based on information of the local window obtained from the input image. When identifying whether the lesion is contained in the image, it is possible to determine whether the lesion is contained in a diagnostic input image based on the pixel values obtained from the single diagnostic input image.

The operation in the learning phase (i.e., learning method) and the execution phase when using the MTANN can be performed using methods in general machine learning, for example, methods in Patent Literatures 4 and 5. That is, a learned model can be constructed by performing supervised machine learning by inputting input data for learning and teacher data into a network configured by the MTANN.

Applicable deep learning techniques are not limited to the MTANN. For example, various deep learning techniques such as convolutional neural networks (CNN), shift-invariant neural networks, deep belief networks (DBN), deep neural networks (DNN), fully convolutional neural networks (FCN), U-Net, V-Net, multi-resolution massive-training artificial neural networks, multiple expert massive-training artificial neural networks, SegNet, VGG-16, LeNet, AlexNet, Resident network (ResNet), Auto encoders and decoders, Generative progressive networks (GAN), Recurrent Neural Networks (RNN), Recursive Neural Networks, Various deep learning techniques such as Long Short-Term Memory (LSTM) can also be used.

By sequentially inputting pairs of the learning data LD and the teacher data TD associated with the learning data LD to perform learning by supervised learning, the machine learning unit 123 optimizes weighting coefficients (or machine learning model parameters) between neurons of a neural network to construct the learned model.

The machine learning unit 123 may output the information INF for constructing the learned model to the estimation unit 121 to the estimation unit 121 (step SL13 in FIG. 10). Thus, the estimator 121 can hold the learned model based on the received information INF (step SL14 in FIG. 10).

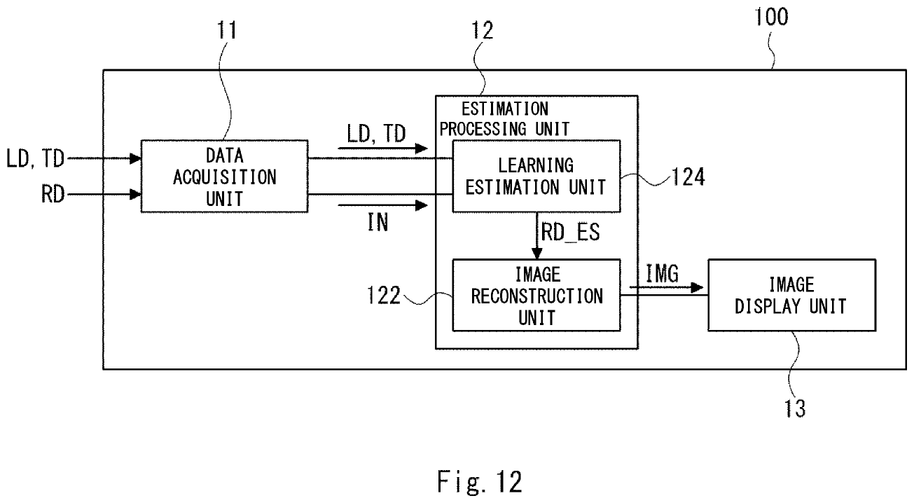

Although the estimation unit 121 and the machine learning unit 123 have been described as separated units, the functions of the estimation unit and the machine learning unit may be integrated without separating them. FIG. 12 schematically shows a configuration of the image processing apparatus 100 when the estimation unit and the machine learning unit are integrated. In FIG. 12, a learning estimation unit 124 integrating the estimation unit 121 and the machine learning unit 123 is provided. In the present configuration, the learned model constructed by machine learning can be used in the estimation phase as it is.

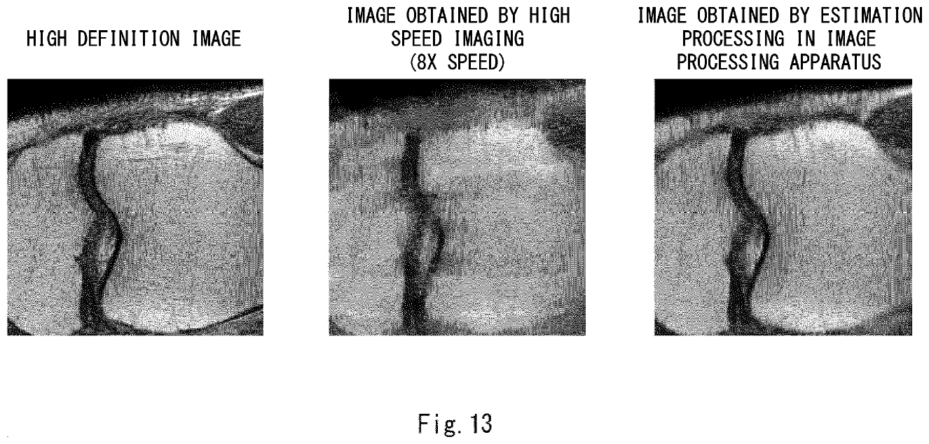

Next, the quality of the image output by the image processing apparatus 100 will be considered. FIG. 13 shows examples of images of the subject when imaged by different imaging methods. In the present drawing, the image on the left side is a high-definition image obtained by reconstructing high-density sampling k-space raw data acquired by imaging the subject with a general imaging method in the MRI apparatus. The center image, which is a comparative example, is a high-speed imaging image obtained by reconstructing lower-density sampling k-space raw data obtained by performing high-speed imaging eight times faster than general high-definition imaging. The image on the right side is a recovered image that displays the recovered image data IMG whose image quality has been recovered, obtained by performing estimation processing on lower-density sampling k-space raw data image obtained by high-speed imaging of the center comparative example by the image processing apparatus 100.

Since the high-speed imaging image of the center comparative example has been imaged at an excessively high-speed, reduced resolution of the reconstructed image, increased artifacts, increased noise, or the like occur. Thus, a tissue boundary is greatly blurred compared to the high-definition image on the left side, and it is particularly difficult to read the condition of joint. Therefore, the image may be considered inappropriate for use in the diagnosis of the subject.

On the other hand, the recovered image on the right side obtained by the image processing apparatus 100 according to the present exemplary embodiment shows sharper enhancement of the tissue compared to the high -speed imaging image in the center. Although the image quality is lower than that of the high-definition image on the left side, the image is clear enough to read the condition of the joint, and it is understandable that the image quality can be used for diagnosis while performing high-speed imaging.

As described above, according to the present configuration, by inputting the image information obtained by high-speed imaging with thinning-out of measurement points, especially by inputting the k-space raw data, into the estimation processing unit and performing the estimation processing, it is possible to obtain a high-definition image more than that obtained by high-speed imaging.

The image thus obtained by the image processing apparatus 100 can be seen as a high-density sample reconstructed image with high resolution, high spatial resolution, good depiction of tissue detail, suppressed noise and artifacts, high signal-to-noise ratio and rich in useful diagnostic information.

In addition, as described above, the imaging time that can be shortened is about ½ when the image quality that can be useful to diagnosis is imaged by a common high-speed imaging technique. However, according to the present configuration, the image quality that can be useful to diagnosis can be obtained from k-space raw data obtained by, for example, 8 times high-speed imaging. Therefore, since the imaging time of the MRI apparatus, which is about a few tens of minutes in general, can be reduced by a factor of several, for example, ⅛, an image that can withstand diagnosis can be obtained in an imaging time of a few minutes.

Therefore, the throughput of the MRI apparatus can be improved significantly more than that of the common high-speed imaging techniques. This also enables the imaging time of the MRI apparatus to be reduced to a few minutes, thus achieving the same throughput as other inspection apparatuses, such as a CT (Computed Tomography) apparatus. It is also possible to greatly reduce a burden on the subject by shortening the imaging time.

### Second Exemplary Embodiment

In the first exemplary embodiment, the image processing apparatus 100 for obtaining the image by reconstructing the estimated k-space raw data RD_ES obtained by inputting the lower-density sampling k-space raw data RD into the estimation processing unit 12 has been described. However, target data is not limited to the k-space raw data, and may be image data obtained by reconstructing from the k-space raw data. Therefore, in the present embodiment, an image processing apparatus that performs estimation processing by inputting image data into an estimation processing unit will be described.

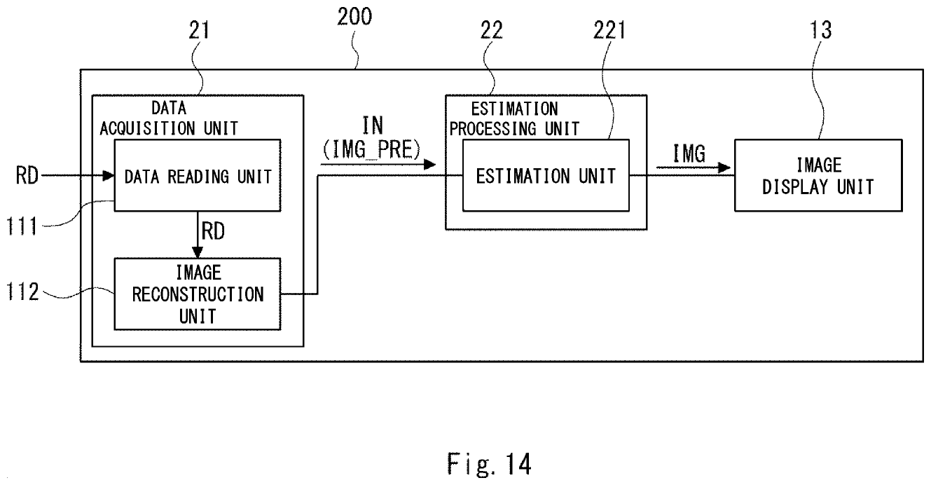

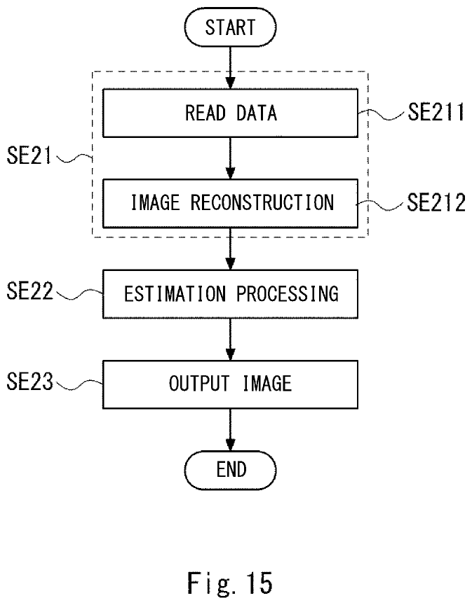

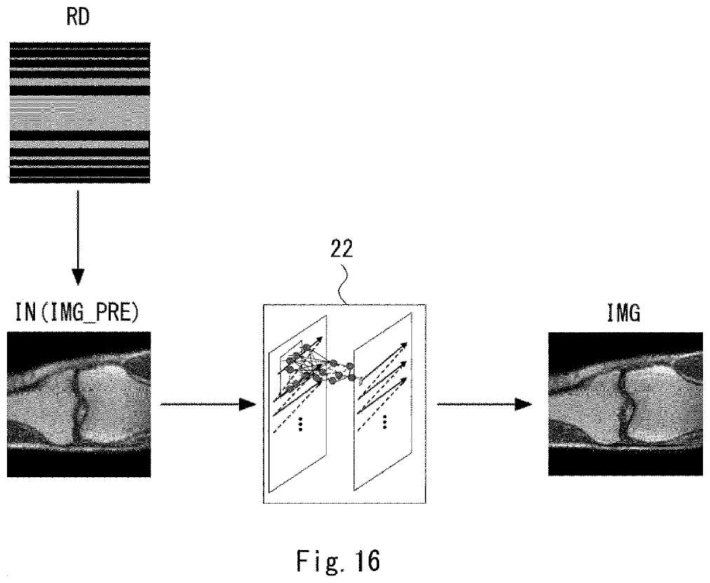

FIG. 14 schematically shows a configuration of an image processing apparatus 200 according to the second exemplary embodiment. FIG. 15 shows a flowchart of processing in an estimation phase of the image processing apparatus 200 according to the second exemplary embodiment. FIG. 16 shows a flow of data in the image processing apparatus 200 according to the second exemplary embodiment. The image processing apparatus 200 has a configuration in which the data acquisition unit 11 and the estimation processing unit 12 of the image processing apparatus 100 according to the first exemplary embodiment are replaced with a data acquisition unit 21 and an estimation processing unit 22, respectively.

The data acquisition unit 21 has a configuration in which the inverse image reconstruction unit 113 is removed from the data acquisition unit 11 of the image processing apparatus 100 in FIG. 6, and performs processing of a step SE21 (steps SE211 and SE212) in FIG. 15. The data reading unit 111 reads lower-density sampling k-space raw data RD (step SE211 in FIG. 15) in the same manner as in the first exemplary embodiment. The image reconstruction unit 112 performs Fourier transforms on the lower-density sampling k-space raw data RD, reconstructs interpolated image data IMG_PRE, and outputs the interpolated image data IMG_PRE as input data IN to the estimation processing unit 22 (step SE212 in FIG. 15). Since the other operations of the data reading unit 111 and the image reconstruction unit 112 are the same as those of the image processing apparatus 100, descriptions thereof will be omitted.

The estimation processing unit 22 includes at least an estimation unit 221. The estimation unit 221 holds a prebuilt learned model, and inputs the interpolated image data IMG_PRE reconstructed as the input data IN to be analyzed to the held learned model, and outputs recovered image data IMG whose image quality is recovered by performing estimation processing (step SE22 in FIG. 15).

The estimation processing unit 22 is also referred to as an image-space estimation processing unit because image data is input as input data to perform estimation processing.

As in the image processing apparatus 100, the image display unit 13 displays an image to be used for diagnosis on a display device such as a display based on the received recovered image data IMG (step SE23 in FIG. 15).

In the above, the configuration and operation (i.e., estimation phase) for estimation and image reconstruction using the learned model in the estimation processing unit 22 have been described, the estimation processing unit 22 may further include a machine learning unit for constructing a learned model.

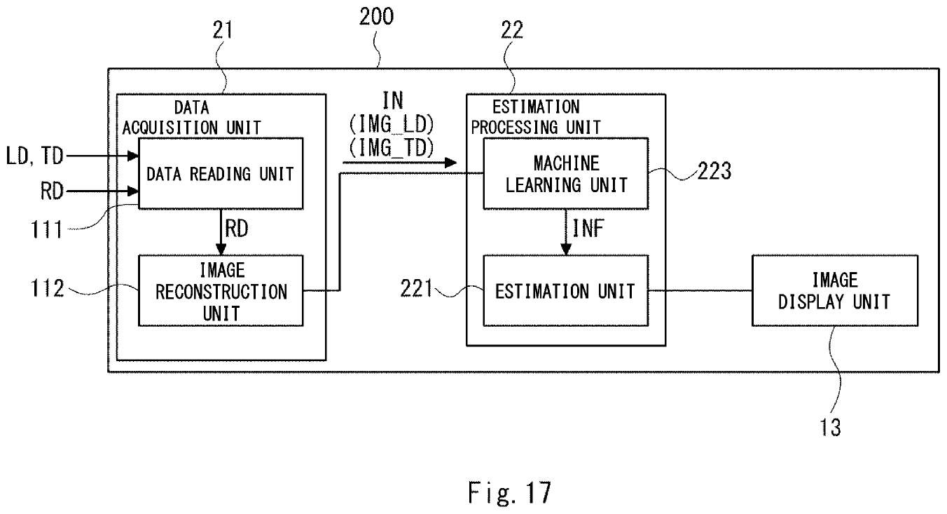

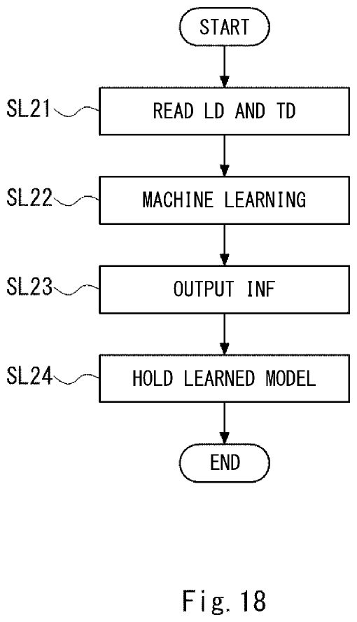

FIG. 17 schematically shows a configuration when the image processing apparatus 200 according to the second exemplary embodiment includes a learned model construction function. FIG. 18 shows a flow chart of learning process of the image processing apparatus 200 according to the second exemplary embodiment. In FIG. 17, the estimation processing unit 22 further includes a machine learning unit 223.

The machine learning unit 223 is configured to learn data read from a memory device by supervised learning in a learning phase. In the learning phase, the data acquisition unit 21 reads learning data LD and teacher data TD imaged in advance by the MRI apparatus and stored in the memory devices such as the RAM 1003 and the memory unit 1008. The data acquisition unit 21 reconstructs the learning data LD and the teacher data TD, which are k-space image data, into learning image data IMG_LD and teacher image data IMG_TD, which are image data, by the image reconstruction unit 212 and outputs the learning image data IMG_LD and the teacher image data IMG_TD to the estimation processing unit 22 (step SL21 in FIG. 18).

The machine learning unit 223 inputs the learning image data IMG_LD and the teacher image data IMG_TD into a neural network to perform machine learning by deep learning (step SL22 in FIG. 18). Note that, also in the present exemplary embodiment, learning can be performed using various deep learning methods including the MTANN.

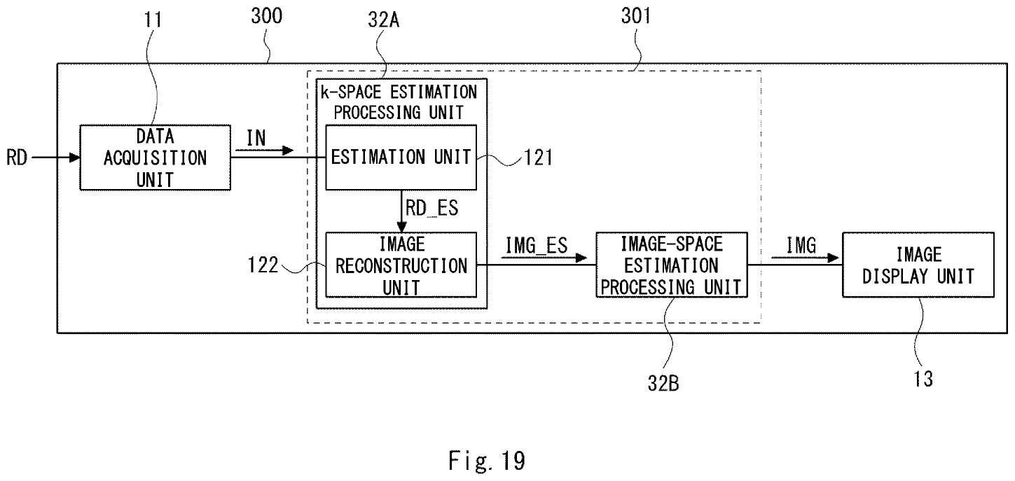

The machine learning unit 223 may output information INF for constructing the learned model to the estimation unit 221 to the estimation unit 221 (step SL23 in FIG. 19). Thus, the estimation unit 221 can hold the learned model based on the received information INF (step SL24 in FIG. 19).

Although not shown in the drawings, as in the first exemplary embodiment, the estimation unit 221 may receive the information INF that has been prepared from an external memory device or the like, or a learning estimation unit integrating the estimation unit and the machine learning unit may be provided for learning and estimation.

Therefore, according to the present configuration, as in the first exemplary embodiment, the throughput of the MRI apparatus can be significantly improved over the general high-speed imaging method. Also, since the imaging time by the MRI apparatus can be reduced to a few minutes, the same throughput as other inspection apparatuses, such as the CT (Computed Tomography) apparatus, can be realized. Moreover, a burden on the subject can be greatly reduced by shortening the imaging time.

### Third Exemplary Embodiment

In first and second exemplary embodiments, examples of performing the estimation processing on the input data only once have been described. However, it is conceivable to improve the image quality of the recovered image data IMG, which is the final output result, by performing estimation processing in multiple stages. In the present embodiment, an image processing apparatus capable of multi-stage estimation processing will be described.

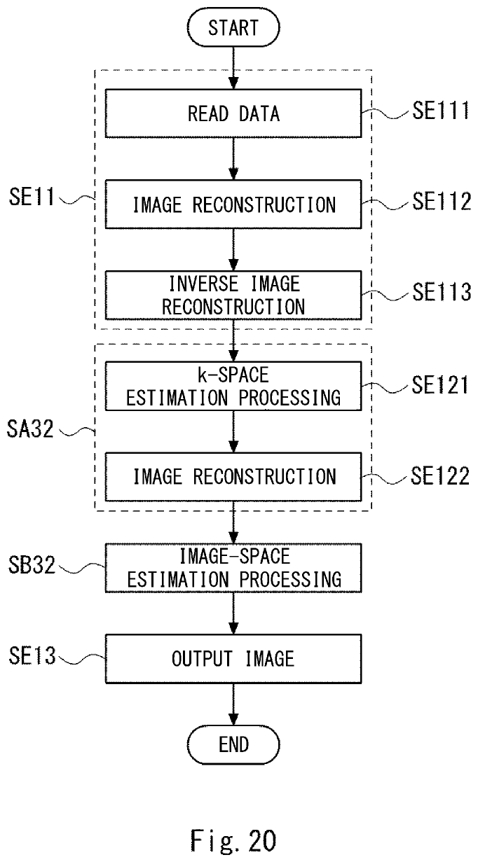

FIG. 19 schematically shows a configuration of an image processing apparatus 300 that is a first configuration example according to the third exemplary embodiment. FIG. 20 shows a flow chart of processing in an estimation phase of the image processing apparatus 300 that is the first configuration example according to the third exemplary embodiment. The image processing apparatus 300 includes the data acquisition unit 11, a k-space estimation processing unit 32A, an image-space estimation processing unit 32B and the image display unit 13.

The k-space estimation processing unit 32A has the same configuration as the estimation processing unit 12 of the image processing apparatus 100 and performs processing of a step SA32 (steps SE121 and SE122) in FIG. 20. In the k-space estimation processing unit 32A, lower-density sampling k-space raw data RD or interpolated k-space raw data RD_PRE, which is the input data IN from the data acquisition unit 11, is input to the estimation unit 121, and the estimation unit 121 outputs estimated k-space raw data RD_ES. The image reconstruction unit 122 reconstructs the estimated k-space raw data RD_ES into estimated image data IMG_ES and outputs the estimated image data IMG_ES to the image-space estimation processing unit 32B (step SA32 in FIG. 20).

The image-space estimation processing unit 32B has the same configuration as the estimation processing unit 22 of the image processing apparatus 200. The image-space estimation processing unit 32B receives the estimated image data IMG_ES as input data and outputs recovered image data IMG that is an estimation result to the image display unit 13 (step SB32 in FIG. 20).

Since the subsequent configuration and operation of the image processing unit 300 are the same as those of the image processing unit 100, descriptions thereof will be omitted.

As described above, according to the image processing unit 300, after a first stage estimation process in the k-space is performed, a second stage estimation process in the image space can be performed. This makes it possible to further improve the image quality of the recovered image data IMG obtained as the final estimation result.

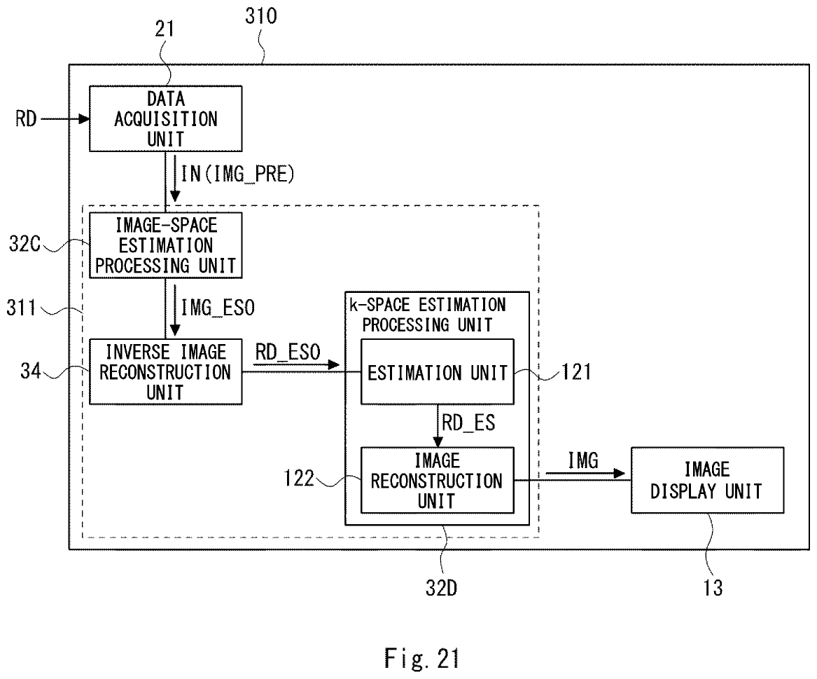

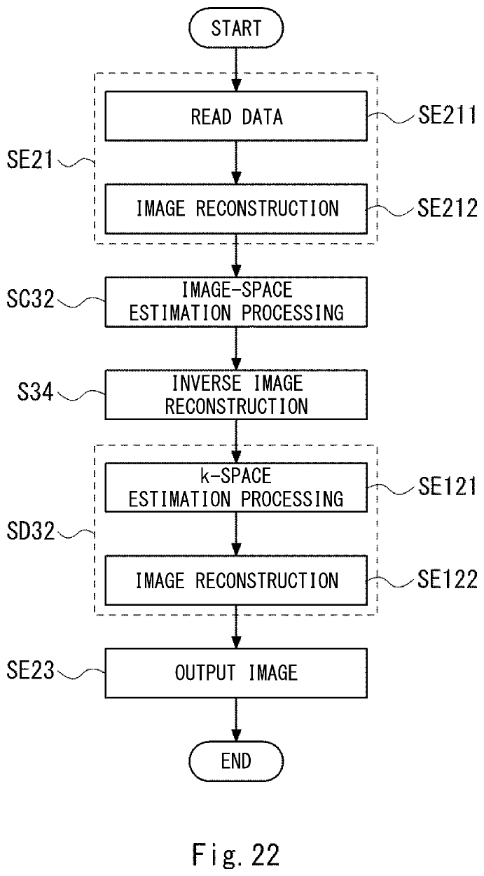

The order of estimation processing is not limited to this example, and after the estimation processing in the image space is performed as a first stage estimation processing, the estimation processing in the k-space may be performed as a second stage estimation processing. FIG. 21 schematically shows a configuration of an image processing apparatus 310 that is a second configuration example according to the third exemplary embodiment. FIG. 22 shows a flow chart of processing in an estimation phase of the image processing apparatus 310 that is the second configuration example according to the third exemplary embodiment. The image processing apparatus 310 includes a data acquisition unit 21, an image-space estimation processing unit 32C, a k-space estimation processing unit 32D, an inverse image reconstruction unit 34, and the image display unit 13.

The image-space estimation processing unit 32C has the same configuration as the estimation processing unit 22 of the image processing apparatus 200. In the image-space estimation processing unit 32C, interpolated image data IMG_PRE is input as input data, and estimated intermediate image data IMG_ES0, which is an estimation result, is output to the inverse image reconstruction unit 34 (step SC32 in FIG. 22).

The inverse image reconstruction unit 34 performs Fourier transforms on the estimated intermediate image data IMG_ES0 to obtain intermediate k-space raw data RD_ES0, and outputs the intermediate k-space raw data RD_ES0 to the k-space estimation processing unit 32D (step S34 in FIG. 22).

The k-space estimation processing unit 32D has the same configuration as the estimation processing unit 12 of the image processing apparatus 100, and performs processing of a step SD32 (steps IMGES0, SE121 and SE122) of FIG. 22. In the k-space estimation processing unit 32D, the intermediate k-space raw data RD_ES0 is input to the estimation unit 121, and the estimation unit 121 outputs the estimated k-space raw data RD_ES that is the estimation result. The image reconstruction unit 122 reconstructs the estimated k-space raw data RD_ES into recovered image data IMG and outputs the recovered image data IMG to the image display unit 13 (step SD32 in FIG. 22).

Since the subsequent configuration and operation of the image processing unit 310 are the same as those of the image processing unit 200, descriptions thereof will be omitted.

As described above, according to the image processing unit 310, after a first stage estimation process in the image space is performed, a second stage estimation process in the k-space can be performed. This makes it possible to further improve the image quality of the recovered image data IMG obtained as the final estimation result.

Note that the two-stage estimation process may be repeated. For example, two or more repetitive estimation processing units 301 configured of the k-space estimation processing unit 32A and the image-space estimation processing unit 32B may be arranged in series between the data acquisition unit 11 and the image display unit 13. Thus, the two-stage estimation processing performed by the image processing unit 300 can be performed multiple times. In this case, to input k-space raw data to each of the repetitive estimation processing units 301, an inverse image reconstruction unit similar to the inverse image reconstruction unit 34 can be inserted between the two adjacent repetitive estimation processing units 301.

Two or more repetitive estimation processing units 311 configured of the image-space estimation processing unit 32C, the k-space estimation processing unit 32D and the inverse image reconstruction unit 34 may be arranged in series between the data acquisition unit 21 and the image display unit 13.

The repetitive estimation processing units 301 and the repetitive estimation processing units 311 may be arranged alternately or randomly in series. When the repetitive estimation processing unit 301 is arranged after the repetitive estimation processing unit 301 or 311, an inverse image reconstruction unit similar to the inverse image reconstruction unit 34 may be inserted between the repetitive estimation processing unit 301 or 311 in the former stage and the repetitive estimation processing unit 301 in the latter stage to input k-space raw data to the repetitive estimation processing unit 301 in the latter stage. When a head repetitive estimation processing unit is the repetitive estimation processing unit 301, input data may be input by the data acquisition unit 11. When the head repetitive estimation processing unit is the repetitive estimation processing unit 311, input data may be input by the data acquisition unit 21.

As described above, according to the image processing apparatus according to the present exemplary embodiment, the image quality of the finally obtained image data can be further improved by performing estimation processing multiple times.

### Fourth Exemplary Embodiment

In the third exemplary embodiment, an example of estimation processing an even number of times for input data has been described. However, estimation processing may be performed three or more odd times. In the present embodiment, an image processing apparatus capable of estimation processing three or more odd times will be described.

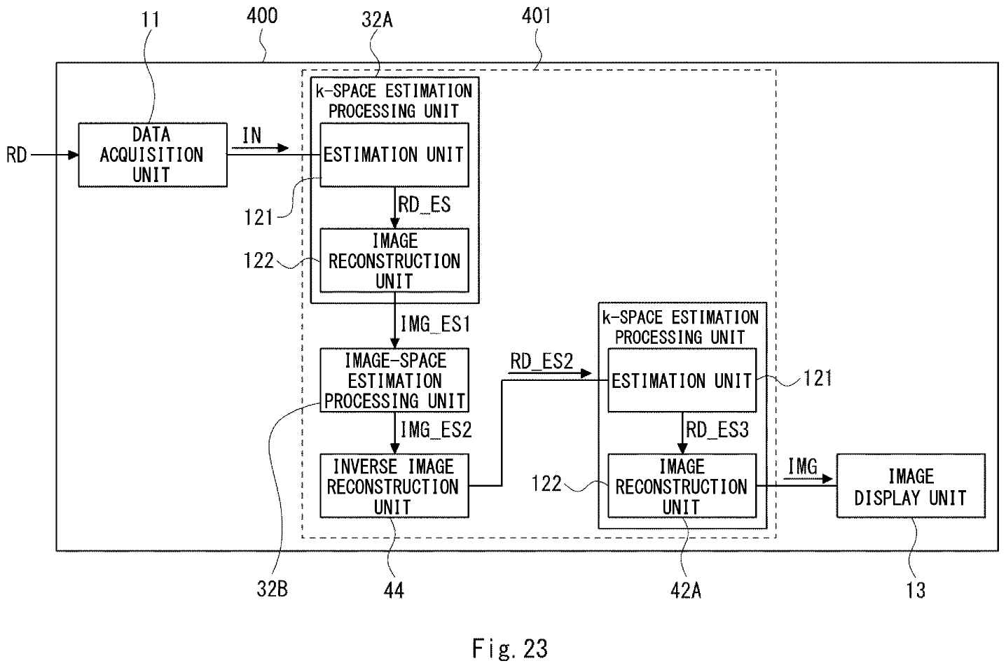

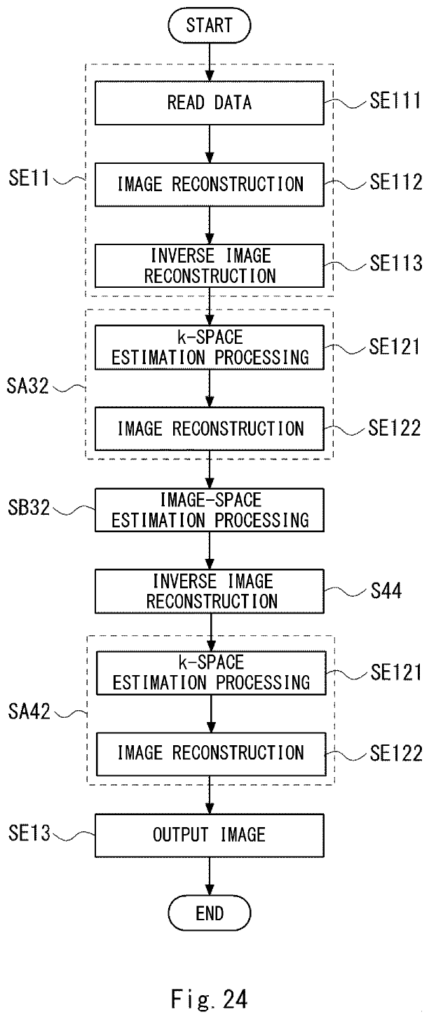

FIG. 23 schematically shows a configuration of an image processing apparatus 400, which is a first configuration example according to a fourth exemplary embodiment. FIG. 24 shows a flow chart of processing in an estimation phase of the image processing apparatus 400, which is the first configuration example according to the fourth exemplary embodiment. The image processing apparatus 400 has a configuration in which an inverse image reconstruction unit 44 and a k-space estimation processing unit 42A are added to the image processing apparatus 300 according to the third exemplary embodiment.

In the present configuration, the k-space estimation processing unit 32A outputs estimated image data IMG_ES1 as an estimation result. The image-space estimation processing unit 32B outputs estimated image data IMG_ES2 to the inverse image reconstruction section 44 as an estimation result corresponding to an estimated image data IMG_ES1 which is input data.

The inverse image reconstruction unit 44 performs Fourier transform on the estimated image data IMG_ES2 to obtain k-space raw data RD_ES2 and outputs the estimated k-space raw data RD_ES2 to the k-space estimation processing unit 42A (step S44 in FIG. 24).

The k-space estimation processing unit 42A has the same configuration as the estimation processing unit 12 of the image processing apparatus 100 and performs processing of a step SA42 (steps SE121 and SE122) in FIG. 24. The estimation unit 121 of the k-space estimation processing unit 42A outputs k-space raw data RD_ES3 which is an estimation result according to the estimated k-space raw data RD_ES2. The image reconstruction unit 122 reconstructs the estimated k-space raw data RD_ES3 into recovered image data IMG that is a final estimation result to the image display unit 13 and outputs the recovered image data IMG (step SA42 in FIG. 22).

Since the subsequent configuration and operation of the image processing apparatus 400 are the same as that of the image processing apparatus 300, descriptions thereof will be omitted.

As described above, according to the image processing apparatus 400, a first stage of estimation processing in the k-space, a second stage of estimation processing in the image space, and a third stage of estimation processing in the k-space can be performed to obtain the recovered image data IMG that is the final estimation result. Therefore, the image quality of the recovered image data IMG can be further improved by the multi-stage estimation processing.

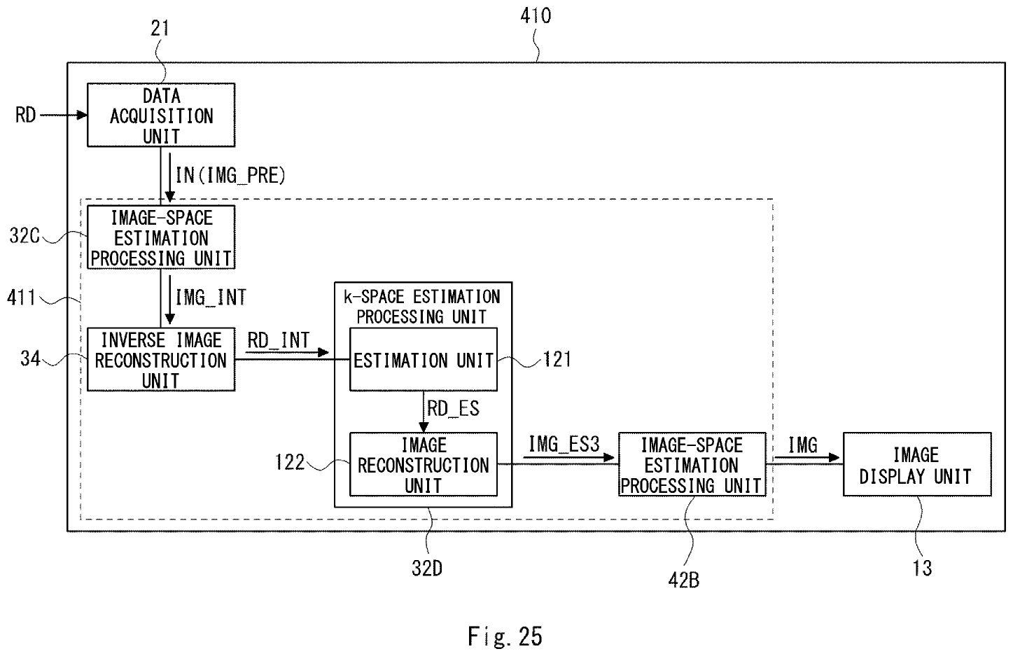

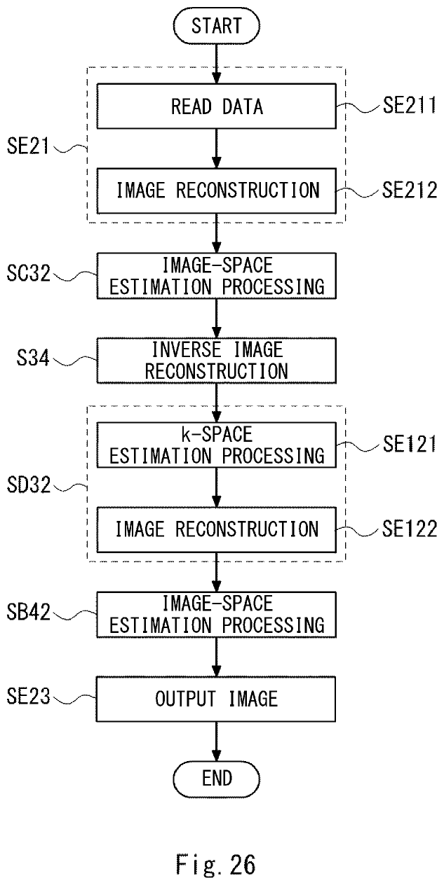

The order of the estimation processing in the k-space and the estimation processing in the image space may be changed. FIG. 25 schematically shows a configuration of an image processing apparatus 410 that is a second configuration example according to the fourth exemplary embodiment. FIG. 26 shows a flowchart of processing in an estimation phase of the image processing apparatus 410 that is the second configuration example according to the fourth exemplary embodiment. The image processing apparatus 410 has a configuration in which an image-space estimation processing unit 42B is added to the image processing apparatus 310 according to the third exemplary embodiment.

In the present configuration, the k-space estimation processing unit 32D outputs estimated image data IMG_ES3 to the image-space estimation processing unit 42B.

The image-space estimation processing unit 42B has the same configuration as the estimation processing unit 22 of the image processing apparatus 200. The image-space estimation processing unit 42B outputs the recovered image data IMG, which is an estimation result corresponding to the estimated image data IMG_ES3, to the image display unit 13 (step SB42 in FIG. 26).

Since the subsequent configuration and operation of the image processing apparatus 410 are the same as those of the image processing apparatus 310, descriptions thereof will be omitted.

Note that two-stage estimation process may be repeated as in the third exemplary embodiment. For example, two or more repetitive estimation processing units 401 configured of the k-space estimation processing unit 32A, the image-space estimation processing unit 32B and the k-space estimation processing unit 42A may be arranged in series between the data acquisition unit 11 and the image display unit 13. Thus, it is also possible to perform three-stage estimation processing performed by the image processing unit 400 multiple times. In this case, an inverse image reconstruction unit similar to the inverse image reconstruction unit 44 may be inserted between two adjacent repetitive estimation processing units 401 to input k-space raw data into each of the repetitive estimation processing units 401.

Two or more repetitive estimation processing units 411 configured of the image-space estimation processing unit 32C, the k-space estimation processing unit 32D and the image-space estimation processing unit 42B may be arranged in series between the data acquisition unit 21 and the image display unit 13.

The repetitive estimation processing units 401 and the repetitive estimation processing units 411 may be arranged alternately or randomly in series. When the repetitive estimation processing unit 401 is arranged after the repetitive estimation processing unit 401 or 411, an inverse image reconstruction unit similar to the inverse image reconstruction unit 44 may be inserted between the repetitive estimation processing unit 401 or 411 in the former stage and the repetitive estimation processing unit 401 in the latter stage to input k -space raw data to the repetitive estimation processing unit 401 in the latter stage. When a head repeat estimation processing unit is the repetitive estimation processing unit 401, input data may be input by the data acquisition unit 11. When the head repetitive estimation processing unit is the repetitive estimation processing unit 411, input data may be input by the data acquisition unit 21.

As described above, according to the image processing apparatus according to the present exemplary embodiment, the image quality of the finally obtained image data can be further improved by performing the estimation processing multiple times, as in the third exemplary embodiment.

### Other Embodiments

Note that the present invention is not limited to the above-described example embodiments, and can be appropriately changed without departing from the gist. For example, the repetitive estimation processing units 301 and 311 according to the third exemplary embodiment and the repetitive estimation processing units 401 and 411 according to the fourth exemplary embodiment may be arranged alternately or randomly. In this case, when the repetitive estimation processing unit 301 or 401 is arranged after the repetitive estimation processing unit 301, 311, 311 or 411, an inverse image reconstruction unit similar to the inverse image reconstruction unit 34 or 44 may be inserted between the repetitive estimation processing unit (repetitive estimation processing unit 301, 311, 301 or 401) arranged in the former stage and the repetitive estimation processing unit (repetitive estimation processing unit 311 or 411) arranged in the latter stage. When a head repetitive estimation processing unit is the repetitive estimation processing unit 301 or 401, input data may be input by the data acquisition unit 11. When the head repetitive estimation processing unit is the repetitive estimation unit 311 or 411, input data may be input by the data acquisition unit 21.

The repetitive processing described in the third and fourth embodiments can be interpreted as follows. That is, a configuration in which one or more k-space estimation processing units and one or more image-space estimation processing units are arranged in series may be used as an estimation processing unit. In this case, when a k-space estimation processing unit is arranged in the latter stage of the k-space estimation processing unit or the image-space estimation processing unit, an inverse image reconstruction unit is provided between the k-space estimation processing unit or the image-space estimation processing unit in the former stage and the k-space estimation processing unit in the latter stage. K-space raw data inversely reconstructed by performing inverse Fourier transform on image data output from the k-space estimation processing unit or the image-space estimation processing unit in the former stage may be output to the k-space estimation processing unit in the latter stage. When a head of serial array of the k-space estimation processing units and the image-space estimation processing units is the k-space estimation processing unit, a data acquisition unit converts thinned-out measurement points of lower-density sampling k-space raw data into interpolated k-space raw data, and outputs the interpolated k-space raw data to the head k-space estimation processing unit as input data. When the head of the serial array of the k-space estimation processing units and the image-space estimation processing units is the image-space estimation processing unit, the data acquisition unit may output interpolated image data obtained by performing Fourier transform on the lower-density sampling k-space raw data to the head image-space estimation processing unit as input data.

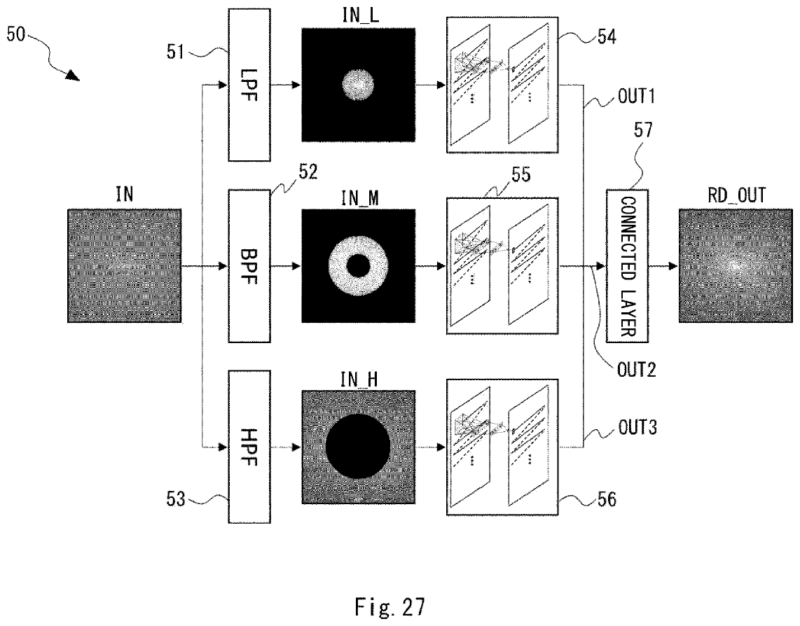

The estimation processing unit described above may be configured as an estimation processing unit of a filter bank image output type in which multiple processing lines with k-space raw data as input data are arranged in parallel. FIG. 27 schematically shows a configuration of an estimation processing unit 50 of a filter bank image output type. The estimation processing unit 50 includes a low-pass filter (LPF) 51, a bandpass filter (BPF) 52, a high-pass filter (HPF) 53, learning estimation units 54 to 56, and a connecting layer 57.

The input data IN is interpolated k-space raw data and is input to the LPF 51, the BPF 52, and the HPF 53. The LPF 51 passes low-frequency band IN_L of the input data IN to the learning estimation unit 54, the BPF 52 passes medium-frequency band IN_M of the input data IN to the learning estimation unit 55, and the HPF 53 passes high-frequency band IN_H of the input data IN to the learning estimation unit 56. Since the k-space is already a frequency space, these frequency-band limiting processes can be realized by multiplying the k-space image by a two-dimensional or three-dimensional function in which the band to be passed is 1 and the other bands to be limited are 0.

The learning estimation units 54 to 56 can perform one or both of estimation processing and learning processing using the frequency components of the input data IN, respectively. K-space raw data OUT1 to OUT3 after the processing output by the learning estimation units 54 to 56 are output to the connecting layer 57. By connecting the raw k-space data OUT1 to OUT3, the connecting layer 57 can output raw k-space data RD_OUT corresponding to the entire frequency range. This makes it possible to output raw k-space data with higher accuracy by performing processing in a learning estimation unit (It goes without saying that it may be an estimation unit or a learning unit.) that performs processing specific to divided frequency bands.

In the above-described exemplary embodiments, the present invention has been mainly described as a hardware configuration, and however, it is not limited to this. Any processing can be achieved by causing a CPU (central processing unit) to execute a computer program. In this case, a computer program can be stored and provided to a computer by use of various types of non-transitory computer-readable media. Such non-transitory computer-readable media include various types of tangible storage media. Examples of such non-transitory computer-readable media include a magnetic storage medium (e.g., a flexible disk, a magnetic tape, a hard-disk drive), a magneto-optical recording medium (e.g., a magneto-optical disk). Additional examples of such non-transitory computer-readable media include a CD-ROM (read-only memory), a CD-R, and a CD-R/W. Yet additional examples of such non-transitory computer-readable media include a semiconductor memory. Examples of a semiconductor memory include a mask ROM, a programmable ROM (PROM), an erasable PROM (EPROM), a flash ROM, or a random-access memory (RAM). A program may also be provided to a computer by use of various types of transitory computer -readable media. Examples of such transitory computer-readable media include an electric signal, an optical signal, and an electromagnetic wave. A transitory computer-readable medium can provide a program to a computer via a wired communication line, such as an electric wire or an optical fiber, or via a wireless communication line.

Although the invention of the present application has been described above with reference to the exemplary embodiments, the invention of the present application is not limited to the above. Various modifications that can be understood by those skilled in the art can be made to the configuration and details of the invention of the present application within the scope of the invention.

This application claims priority based on Japanese Patent Application No. 2021-32652 filed on Mar. 2, 2021 and the disclosure of which is incorporated herein in its entirety.

### REFERENCE SIGNS LIST

1 MTANN
2 INPUT LAYER
3 HIDDEN LAYER
4 OUTPUT LAYER
11,21 DATA ACQUISITION UNIT
12, 22, 50 ESTIMATION PROCESSING UNIT
13 IMAGE DISPLAY UNIT
32A, 32D, 42A K-SPACE ESTIMATION PROCESSING UNIT
32B, 32C, 42B IMAGE-SPACE ESTIMATION PROCESSING UNIT
34, 44, 113 INVERSE IMAGE RECONSTRUCTION UNIT
51 LPF
52 BPF
53 HPF
54 TO 56, 124 LEARNING ESTIMATION UNIT
57 CONNECTING LAYER
100, 200, 300, 310, 400, 410 IMAGE PROCESSING UNIT
111 DATA READING UNIT
112, 122 IMAGE RECONSTRUCTION UNIT
121, 221 ESTIMATION UNIT
123, 223 MACHINE LEARNING UNIT
301, 311, 401, 411 REPETITIVE ESTIMATION UNIT
1000 COMPUTER
1001 CPU
1002 ROM
1003 RAM
1004 BUS
1005 INPUT/OUTPUT INTERFACE
1006 NPUT UNIT
1007 OUTPUT UNIT
1008 MEMORY UNIT
1009 COMMUNICATION UNIT
1010 DRIVE
1011 MAGNETIC DISK
1012 OPTICAL DISK
1013 FLEXIBLE DISK
1014 SEMICONDUCTOR MEMORY
IMG_ES, IMG_ES1 TO IMG_ES3 ESTIMATED IMAGE DATA
IMG_ES0 ESTIMATED INTERMEDIATE IMAGE DATA
IMG_PRE INTERPOLATED IMAGE DATA
IMG_LD, LD LEARNING IMAGE DATA
IMG_TD, TD TEACHER IMAGE DATA
IN INPUT DATA
INF INFORMATION
LD LEARNING DATA
RD OW-DENSITY SAMPLING K-SPACE RAW DATA
RD_ES, RD_ES2, RD_ES3 ESTIMATED K-SPACE RAW DATA
RD_ES0 INTERMEDIATE K-SPACE RAW DATA
RD_PRE INTERPOLATED K-SPACE RAW DATA

1. An image processing apparatus comprising:
a data acquisition unit configured to acquire lower-density sampling k-space raw data obtained by imaging a subject by thinning-out measurement points by an MRI apparatus and output input data based on the lower-density sampling k-space raw data;
an estimation processing unit configured to perform estimation processing by inputting the input data into a learned model and output recovered image data whose image quality reduced by the thinned-out measurement points has been recovered; and
an image display unit configured to display the recovered image data.

2. The image processing apparatus according to claim 1, wherein
the data acquisition unit converts the lower-density sampling k-space raw data into interpolated k-space raw data by interpolating the thinned-out measurement points and outputs the interpolated k-space raw data to the estimation processing unit as the input data, and
the estimation processing unit is configured as a k-space estimation processing unit configured to perform estimation processing by inputting the interpolated k-space raw data into the learned model, output estimated k-space raw data whose image quality reduced by the thinned-out measurement points has been recovered, and output the recovered image data reconstructed by performing Fourier transform on the estimated k-space raw data.

3. The image processing apparatus according to claim 2, wherein the data acquisition unit acquires the interpolated k-space raw data inversely reconstructed by performing inverse Fourier-transform on image data that has been reconstructed by performing Fourier-transform on the lower-density sampling k-space raw data.

4. The image processing apparatus according to claim 1, wherein
the data acquisition unit outputs image data obtained by performing Fourier transform on the lower-density sampling k-space raw data to the estimation processing unit as the input data, and
the estimation processing unit is configured as an image-space estimation unit configured to perform estimation processing by inputting the transformed image data into the learned model and to output the recovered image data whose image quality reduced by the thinned-out measurement points has been recovered.

5. The image processing apparatus according to claim 1, wherein
in the estimation processing unit, one or more k-space estimation processing units and one or more image-space estimation processing units are arranged in series,
the k-space estimation processing unit performs estimation processing by inputting k-space raw data, which is input data, into the learned model, outputs the k-space raw data whose image quality has been recovered, and outputs second image data reconstructed by performing Fourier transform on the k-space raw data,
the image-space estimation processing unit performs estimation processing by inputting the image data, which is input data, into the learned model, and outputs image data whose image quality has been recovered,
when the k-space estimation processing unit is arranged in the latter stage of the image-space estimation processing unit, an inverse image reconstruction unit is provided between the image-space estimation processing unit and the k-space estimation processing unit to output k-space raw data, which is inversely reconstructed by performing inverse Fourier transform on image data output from the image-space estimation processing unit in the former stage, to the k-space estimation processing unit in the latter stage, and
image data output from the end of serial array of the k-space estimation processing unit and the image-space estimation processing unit is output as the recovered image data.

6. The image processing apparatus according to claim 5, wherein
when a head of the serial array of the k-space estimation processing unit and the image-space estimation processing unit is the k-space estimation processing unit, the data acquisition unit converts the lower-density sampling k-space raw data into interpolated k-space raw data by interpolating the thinned-out measurement points and outputs the interpolated k-space raw data to the head k-space estimation processing unit as the input data, and
when the head of the serial array of the k-space estimation processing unit and the image-space estimation processing unit is the image-space estimation processing unit, the data acquisition unit outputs image data obtained by performing Fourier transform on the lower-density sampling k-space raw data to the head image-space estimation processing unit as the input data.

7. The image processing apparatus according to any one of claims 1 to 6, wherein
the data acquisition unit reads learning data that is lower-density sampling k-space raw data obtained in advance by imaging the subject with thinning-out the measurement points by the MRI apparatus and teacher data that is high-density sampling k-space raw data obtained in advance by imaging the subject without thinning-out the measurement points by the MRI apparatus, and outputs learning input data based on the learning data and the teacher data that have been read to the estimation processing unit, and
the estimation processing unit constructs the learned model by performing supervised learning according to the learning input data.

8. The image processing apparatus according to any one of claims 1 to 6, wherein
the data acquisition unit reads learning data obtained by reconstructing lower-density sampling k-space raw data obtained in advance by imaging the subject with thinning-out the measurement points by the MRI apparatus and teacher data obtained by reconstructing high-density sampling k-space raw data obtained in advance by imaging the subject without thinning-out the measurement points by the MRI apparatus, and outputs learning input data based on the learning data and the teacher data that has been read to the estimation processing unit, and
the estimation processing unit constructs the learned model by performing supervised learning based on the learning input data.

9. The image processing apparatus according to any one of claims 1 to 8, wherein the learned model is a network constructed by inputting learning data and teacher data into a network configured as a MTANN (Massive-Training Artificial Neural Network) to perform learning.

10. An image processing method comprising:
acquiring lower-density sampling k-space raw data obtained by imaging a subject with thinning-out measurement points by an MRI apparatus, and outputting input data based on the lower-density sampling k-space raw data;
performing estimation processing by inputting the input data into a learned model, and outputting recovered image data whose image quality reduced by the thinned-out measurement points has been recovered; and
displaying the recovered image data.

11. A non-transitory computer-readable medium storing a program to cause a computer to execute:
acquiring lower-density sampling k-space raw data obtained by imaging a subject with thinning-out measurement points by an MRI apparatus, and outputting input data based on the lower-density sampling k-space raw data;
performing estimation processing by inputting the input data into a learned model, and outputting recovered image data whose image quality reduced by the thinned-out measurement points has been recovered; and
displaying the recovered image data.
